(19) United States
(12) Patent Application Publication
(10) Pub. No.: US 2025/0228449 A1
(43) Pub. Date: Jul. 17, 2025

(54) PATHOLOGY AND/OR EYE-SIDED DEPENDENT ILLUMINATION FOR RETINAL IMAGING

(71) Applicant: Verily Life Sciences LLC, Dallas, TX (US)

(72) Inventors: Sam Kavusi, Menlo Park, CA (US); Alireza Dastgheib, Mountain View, CA (US)

(21) Appl. No.: 18/853,905

(22) PCT Filed: Feb. 23, 2023
(86) PCT No.: PCT/US2023/013737

§ 371 Date: Oct. 3, 2024

Related U.S. Application Data
(60) Provisional application No. 63/345,258, filed on May 24, 2022.

(51) Int. Cl. A61B 3/00; A61B 3/12; A61B 3/15; H04N 23/56; H04N 23/60; H04N 23/611; H04N 23/63; H04N 23/74

(52) U.S. Cl. CPC A61B 3/0025; A61B 3/12; A61B 3/152; H04N 23/56; H04N 23/611; H04N 23/635; H04N 23/64; H04N 23/74

(57) ABSTRACT

A retinal imaging system includes an eyepiece lens assembly, an image sensor adapted to acquire a retinal image of an eye through the eyepiece lens assembly, and a controller communicatively coupled to the image sensor. The controller including logic that when executed causes the retinal imaging system to perform operations including: obtaining an indication of a pathology of interest (POI) related to the eye or an eye sidedness, selecting an eyebox location for an eyebox of the retinal imaging system based at least in part on the POI or an eye sidedness, and acquiring the retinal image of the eye when the eye is determined to be positioned within the eyebox. The eyebox corresponds to a bound region in space defined relative to the eyepiece lens assembly.

### CROSS-REFERENCE TO RELATED APPLICATIONS

This application claims the benefit of U.S. Application No. 63/345,258, filed on May 24, 2022, the contents of which are incorporated herein by reference.

### TECHNICAL FIELD

This disclosure relates generally to retinal imaging technologies, and in particular but not exclusively, relates to illumination techniques for retinal imaging.

### BACKGROUND INFORMATION

Retinal imaging is a part of basic eye exams for screening, field diagnosis, and progress monitoring of many retinal diseases. A high-fidelity retinal image is important for accurate screening, diagnosis, and monitoring. Bright illumination of the posterior interior surface of the eye (i.e., retina) through the pupil improves image fidelity but often creates optical aberrations or image artifacts, such as corneal reflections, iris reflections, lens flare, haze, or pupillary shadows, if the retinal camera and illumination source are not appropriately aligned with the eye. Simply increasing the brightness of the illumination does not overcome these problems, but rather makes the optical artifacts more pronounced, which undermines the goal of improving image fidelity.

Accordingly, camera alignment is very important, particularly with conventional retinal cameras, which typically have a limited eyebox due to the need to block the deleterious image artifacts listed above. Referring to FIG. 1A, the eyebox 100 for a retinal camera 105 is a bound region in three-dimensional space typically defined relative to an eyepiece 110 of the retinal camera 105 and within which a specific portion (e.g., center) of a pupil 115 or cornea of the eye should reside to acquire an acceptable image of the retina. The small size of conventional eyeboxes makes retinal camera alignment difficult and patient interactions during the alignment process often strained.

A conventional retinal camera system (such as retinal camera 105) use a single eyebox 100 having a single location (defined relative to the eyepiece lens 110 of the camera system) from which both the left-side and right-side eyes are imaged. However, this single location is a compromise location that is not optimized for the individual eye and furthermore does not account for the need to obtain higher quality images in specific regions of interest within the left and/or right eyes to help the doctor screen, diagnose, monitor, or treat specific ophthalmic pathologies.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIGS. 1A and 1B (PRIOR ART) illustrate a single eyebox used by a conventional retinal imaging system to inspect both the left-side and right-side eyes.

FIG. 2A illustrates a retinal image of a left-sided eye with various image artifacts, in accordance with an embodiment of the disclosure.

FIG. 2B illustrates a retinal image of a right-sided eye without image artifacts, in accordance with an embodiment of the disclosure.

FIGS. 3A and 3B illustrate moving the eyebox of a retinal imaging system to different locations dependent upon eye sidedness and/or pathologies of interest (POI), in accordance with an embodiment of the disclosure.

FIG. 4 illustrates a retinal imaging system capable of using a dynamic eyebox location, in accordance with an embodiment of the disclosure.

FIG. 5 illustrates a dynamic ring illuminator for illuminating a retina during retinal imaging, in accordance with an embodiment of the disclosure.

FIG. 6 is a flow chart illustrating a process for capturing retinal images using dynamic eyebox locations, fixation targets, and illumination patterns based upon eye sidedness and/or POI, in accordance with an embodiment of the disclosure.

FIGS. 7A & 7B illustrate a dynamic fixation target including an eyebox reference and an eye location reference that coax the eye into a specific eyebox and alignment, in accordance with an embodiment of the disclosure.

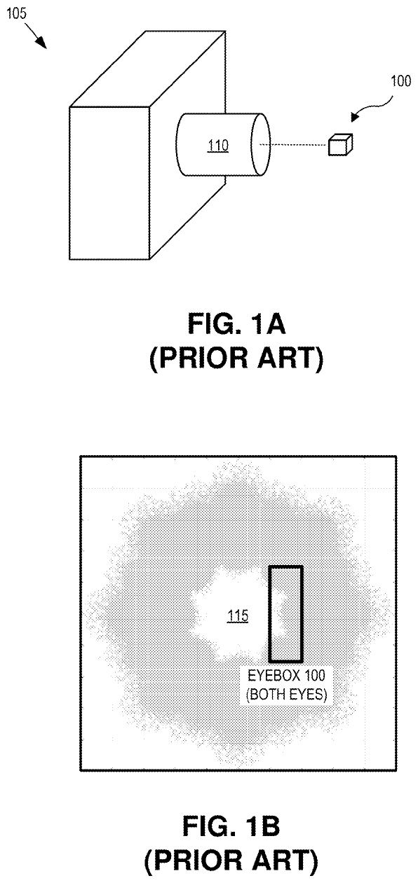

### DETAILED DESCRIPTION

Embodiments of an apparatus, system, and method of operation for a retinal imaging system that adapts the eyebox location based upon pathologies of interest (POI) and/or eye sidedness are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

High fidelity retinal images are important for screening, diagnosing, and monitoring many retinal diseases. To this end, reducing or eliminating instances of image artifacts that occlude, or otherwise malign portions of the retinal image is desirable. This can be particularly true when specific regions of interest in a particular eye (e.g., right-sided eye or left-sided eye) need to be clearly imaged to screen for, diagnosis, monitor, or treat a specific ophthalmic pathology. Conventional retinal imaging systems use an eyebox in a fixed eyebox location that is not only fixed for a given eye, but also fixed across both the left-side and right-side eyes.

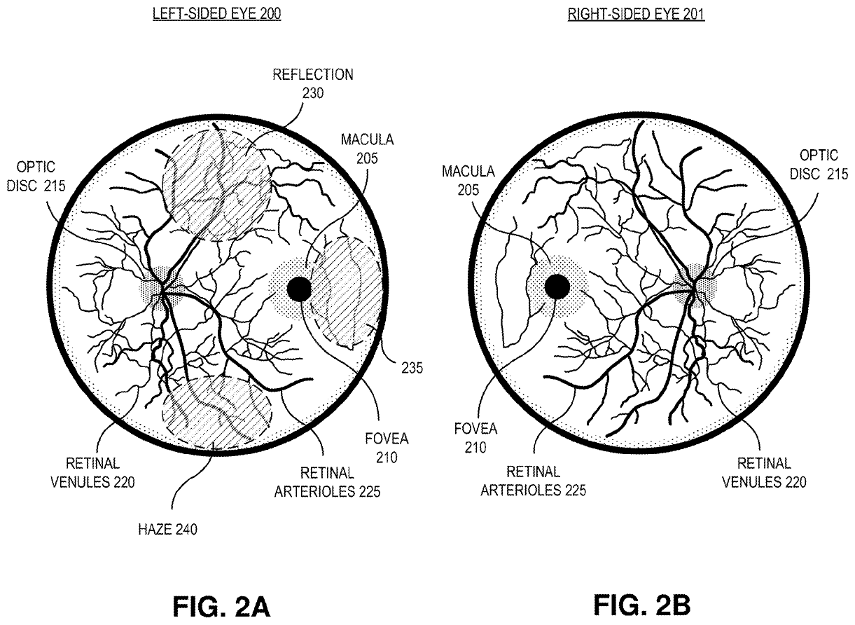

FIGS. 2A and 2B illustrate example retinal images of a left-sided eye 200 and a right-sided eye 201 (as seen from the perspective of the doctor or retinal imaging system). As seen in FIGS. 2A and 2B, the anatomical features (e.g., macula 205, fovea 210, optic disc 215, retinal venules 220, and retinal arterioles 225) are flipped about a vertical axis, changing the relative position of these anatomical features between each eye 200 and 201. As such, the critical anatomical features of interest in each eye 200 or 201 have different positions depending upon the eye sidedness. If a high-quality image of a particular anatomical feature is desired, then the optimal eyebox location and illumination pattern may not be the same between each eye 200 and 201. Similarly, different pathologies require good visualization of different portions of the eye. For example, diagnosis and treatment of diabetic retinopathy is well served with good visualization of macula 205 while diagnosis and treatment of glaucoma is well served with good visualization of optic disc 215. Accordingly, retinal imaging for the purposes of screen, diagnosing, monitoring, and treating ophthalmic pathologies can also be helped by adjusting the eyebox location and illumination patterns based upon a POI to ensure the best possible image of the pertinent retinal region.

The desirability of dynamically selected eyebox location and/or illumination patterns is further highlighted by FIG. 2A. As illustrated, retinal imaging can suffer from multiple image artifacts including a reflection 230, a pupillary shadow 235, a haze 240, or otherwise. These image artifacts may arise when less than optimal alignment between the retinal imaging system and the eye permit stray light and deleterious reflections from the illumination source to enter the imaging path and ultimately are captured by the image sensor with the retinal image light. Misalignment (or nonoptimal alignment) can lead to deleterious corneal/iris reflections, refractive scattering from the crystalline lens, occlusion of the imaging aperture, optical aberrations due to off axis passage through the crystalline lens, the blockage of imaging light by the iris, and/or other issues. If these image artifacts occlude the region of interest particularly relevant for screening, diagnosing, or treating a POI, then diseases may go unnoticed or improperly treated. Accordingly, selective placement of the eyebox and customized illumination patterns based upon eye sidedness and/or POI, can improve retinal images by placing these artifacts in less relevant locations and/or reducing or eliminating the artifacts entirely. In some instances, an eyebox location that achieves 100% removal of an image artifact isn't readily or easily achievable. In such instances, the dynamic fixation targets that encourage the eye to move to multiple different eyebox locations, using multiple different illumination patterns, may encourage the patient's eye to roll or align to multiple different directions/locations. Although the particular image artifact may not be entirely removed from all or any of the multiple retinal images, the patient's eye is directed to roll/move in such a manner that each region of interest in the retina is clearly imaged in at least one retinal image. The multiple retinal images may then be combined or stacked to entirely remove image artifacts from a composite retinal image, at least in the regions of interest that are pertinent to the eye sidedness and/or POI.

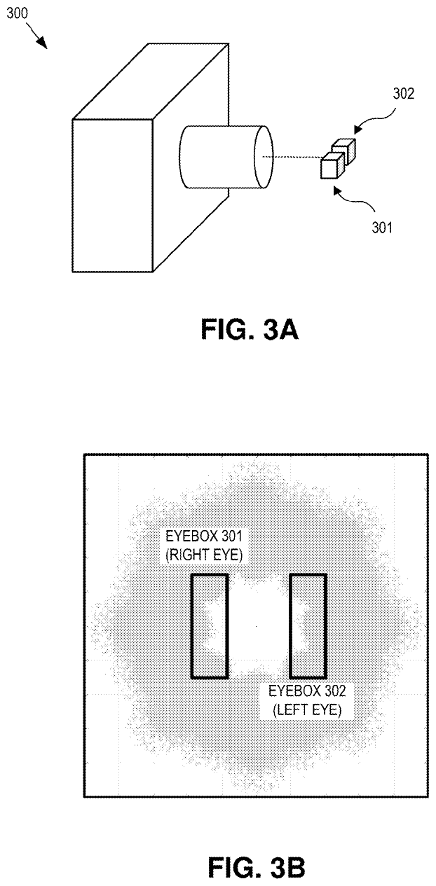

FIGS. 3A and 3B illustrate moving the eyebox of a retinal imaging system 300 to different locations dependent upon eye sidedness and/or POI, in accordance with an embodiment of the disclosure. As illustrated, retinal imaging of the right-sided eye may be well served using an eyebox 301 that is offset to the left (e.g., shifted left by 1.5 mm from center) while retinal imaging of the left-sided eye may be well served using an eyebox 302 that is offset to the right (e.g., shifted right by 1.5 mm from center). These eye sidedness offsets can improve visualization of the important anatomical features. This selective adjustment of the eyebox location can also be indexed to POI (or a combination of POI and eye sidedness) where inspection of specific retinal regions are of particular interest for screening, diagnosing, and treating a given ophthalmic pathology. Although FIGS. 3A and 3B use cuboids or rectangles to illustrate eyeboxes 301 and 302, it should be appreciated that the eyeboxes may assume a variety of different three-dimensional shapes (e.g., sphere, ellipsoid, etc.).

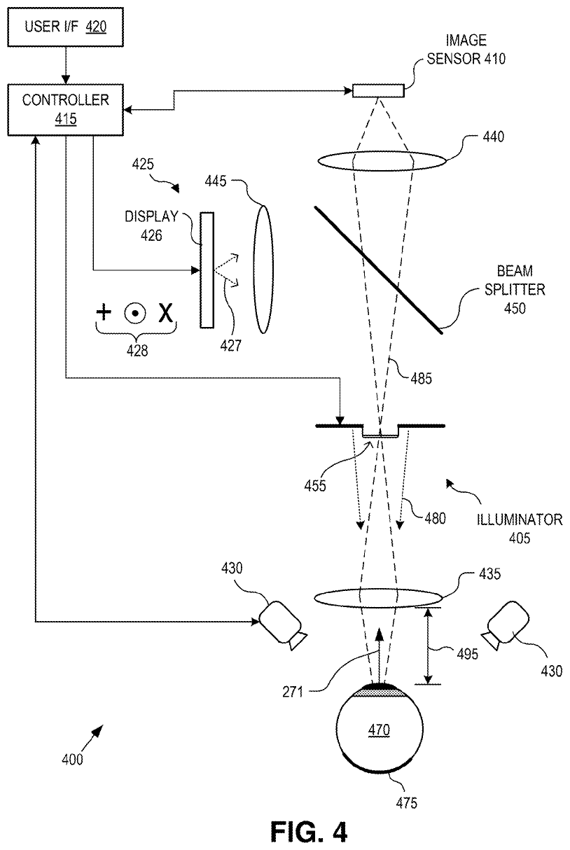

FIG. 4 illustrates a retinal imaging system 400 capable of using a dynamic eyebox location based upon POI and/or eye sidedness, in accordance with an embodiment of the disclosure. The illustrated embodiment of retinal imaging system 400 includes an illuminator 405, an image sensor 410 (also referred to as a retinal image sensor), a controller 415, a user interface 420, a dynamic fixation target 425, an alignment tracking camera system 430, and an optical relay system. The illustrated embodiment of the optical relay system includes lens assemblies 435, 440, 445 and a beam splitter 450. Lens assembly 435 may also be referred to as an eyepiece lens assembly 435. The illustrated embodiment of illuminator 405 comprises a dynamic ring illuminator with a center aperture 455. The illustrated embodiment of dynamic fixation target 425 includes a display 426 that outputs a dynamic fixation image 427 that may include one or more reference markers 428 that represent relative eyebox and/or eye locations.

The optical relay system serves to direct (e.g., pass or reflect) illumination light 480 output from illuminator 405 along an illumination path through the pupil of eye 470 to illuminate retina 475 while also directing image light 485 of retina 475 (i.e., the retinal image) along an imaging path to image sensor 410. Image light 485 is formed by the scattered reflection of illumination light 480 off of retina 475. In the illustrated embodiment, the optical relay system further includes beam splitter 450, which passes at least a portion of image light 485 to image sensor 410 while also optically coupling dynamic fixation target 425 to eyepiece lens assembly 435 and directing dynamic fixation image 427 output from display 226 to eye 470. Beam splitter 450 may be implemented as a polarized beam splitter, a non-polarized beam splitter (e.g., 90% transmissive and 10% reflective, 50/50 beam splitter, etc.), a multi-layer dichroic beam splitter, or otherwise. The optical relay system includes a number of lenses, such as lenses 435, 440, and 445, to focus the various light paths as needed. For example, lens 435 may include one or more lensing elements that collectively form an eyepiece lens assembly that is displaced from the cornea of eye 470 by an eye relief 495 during operation. Lens 440 may include one or more lens elements for bring image light 485 to a focus on image sensor 410. Lens 445 may include one or more lens elements for focusing dynamic fixation image 427. It should be appreciated that optical relay system may be implemented with a number and variety of optical elements (e.g., lenses, reflective surfaces, diffractive surfaces, etc.) and may vary from the configuration illustrated in FIG. 4.

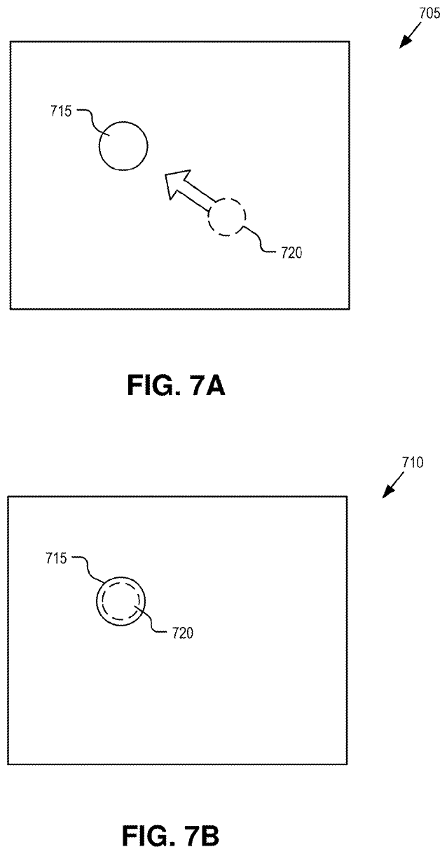

In one embodiment, dynamic fixation image 427 output from display 426 represents a point of fixation upon which the patient can accommodate their focus and fix their gaze. The dynamic fixation image 427 may be an image of a plus-sign, a bullseye, a cross, a target, circles, or other shape or collection of shapes (e.g., see FIGS. 7A and 7B). In the illustrated embodiment, dynamic fixation target 425 is implemented as virtual images output from a display 426. However, the point of fixation may be implemented in a variety of other ways including physical target(s) that are actuated or optically manipulated. Dynamic fixation target 425 not only can aid with obtaining alignment between retinal imaging system 400 and eye 470 by providing visual feedback to the patient, but may also give the patient a fixation point/target upon which the patient can accommodate and stabilize their vision. The dynamic fixation target may be moved by translating the image of the fixation target (e.g., reference markers 428) about display 426 as desired (e.g., moving a symbol or image up/down or left/right on display 426). Display 426 may be implemented with a variety of technologies including a liquid crystal display (LCD), light emitting diodes (LEDs), various illuminated shapes (e.g., an illuminated cross or concentric circles), or otherwise. Of course, the dynamic fixation target may be implemented in other manners than a virtual image on a display. For example, the dynamic fixation target may be a physical object (e.g., crosshairs, etc.) that is physically manipulated.

Controller 415 is coupled to image sensor 410, display 426, illuminator 405, and alignment tracking camera system 430 to orchestrate their operation. Controller 415 may include software/firmware logic executing on a microcontroller, hardware logic (e.g., application specific integrated circuit, field programmable gate array, etc.), or a combination of software and hardware logic. Although FIG. 4 illustrates controller 415 as a distinct functional element, the logical functions performed by controller 415 may be decentralized across a number hardware elements. Controller 415 may further include input/output (I/O) ports, communication systems, or otherwise. Controller 415 is coupled to user interface 420 to receive user input and provide user control over retinal imaging system 400. User interface 420 may include one or more buttons, dials, joysticks, feedback displays, indicator lights, etc.

Image sensor 410 may be implemented using a variety of imaging technologies, such as complementary metal-oxide-semiconductor (CMOS) image sensors, charged-coupled device (CCD) image sensors, or otherwise. In one embodiment, image sensor 410 includes an onboard memory buffer or attached memory to store/buffer retinal images. In one embodiment, image sensor 410 may include an integrated image signal processor (ISP) to permit highspeed digital processing of retinal images buffered in the onboard memory. The onboard image buffer and ISP may facilitate high frame rate image burst captures, image processing, image stacking, and output of high-quality composite retinal images. The integrated ISP may be considered a decentralized component of controller 415.

Alignment tracking camera system 430 operates to track lateral alignment (or misalignment) and relief offset between retinal imaging system 400 and eye 470, and in particular, between eyepiece lens assembly 435 and eye 470. System 430 may operate using a variety of different techniques to track the relative position of eye 470 to retinal imaging system 400 including pupil tracking or iris tracking. In the illustrated embodiment, system 430 includes two cameras disposed on either side of eyepiece lens assembly 435 to enable triangulation and obtain X, Y, and Z gross position information about the pupil or iris. In one embodiment, system 430 also includes one or more infrared (IR) emitters to track eye 470 with IR light while retinal images are acquired with bursts of visible spectrum light output through eyepiece lens assembly 435 from illuminator 405. In such an embodiment, IR filters may be positioned within the image path to filter the IR tracking light. In some embodiments, the tracking illumination is temporally offset from image acquisition with white light bursts.

Lateral eye alignment may be measured via retinal images acquired by image sensor 410, or separately/additionally, by system 430. In the illustrated embodiment, system 430 is positioned externally to view eye 470 from outside of eyepiece lens assembly 435. In other embodiments, system 430 may be optically coupled via the optical relay components to view and track eye 470 through eyepiece lens assembly 435.

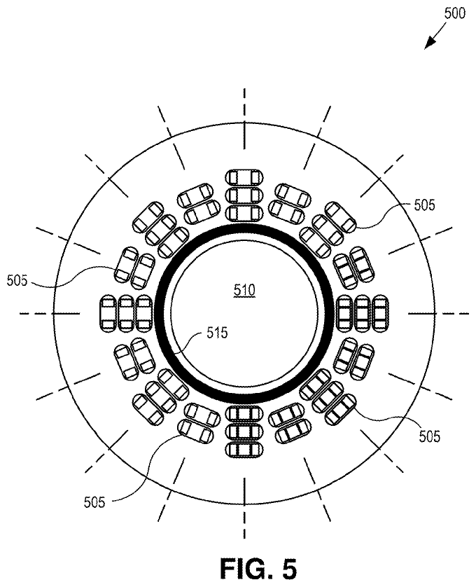

FIG. 5 illustrates a dynamic ring illuminator 500 for illuminating retina 475 during retinal imaging, in accordance with an embodiment of the disclosure. Dynamic ring illuminator 500 represents one possible implementation of illuminator 405. The illustrated embodiment of dynamic ring illuminator 500 includes a ring of light sources 505 surrounding a central aperture 510, which is also encircled by a baffle 515 (e.g., cone, light shade, etc.) that blocks stray or off axis light. Light sources 505 are disposed around central aperture 510 with varying radial and axial offsets. Light sources 505 may be independently activated by controller 415 to emit selective illumination patterns. These patterns may be selected based upon an eye position relative to eyepiece lens 435 as determined by pupil/iris tracking using system 430 or by retinal tracking using image sensor 410. The patterns may also be selected dependent upon eye-sidedness and/or POI. In the illustrated embodiment, each light source 505 includes two emitters-a white light emitter for color retinal images and an IR emitter for IR images. Since the human eye has little to no response to IR light, the IR emitters may be used for eye tracking and acquisition of preliminary images (e.g., determination of eye-sidedness) prior to acquiring full color burst images. The white light emitters may burst white light for a couple hundred milliseconds permitting acquisition of a sequence of full color images before the physiological response causes a blink and/or too much pupilar constriction.

Returning to FIG. 4, during operation, controller 415 operates illuminator 405 and retinal image sensor 410 to capture one or more retinal images. Illumination light 480 is directed through the pupil of eye 470 to illuminate retina 475. The scattered reflections from retina 475 are directed back along the image path through aperture 455 to image sensor 410. When eye 470 is properly aligned within the selected eyebox of system 400, aperture 455 operates to block deleterious reflections and light scattering that would otherwise malign the retinal image while passing the image light itself. Prior to capturing the retinal image, controller 415 operates display 426 to output a dynamic fixation image 427 to guide the patient's gaze. One or more initial or preliminary eye images (e.g., initial alignment images), either from image sensor 410 or alignment tracking camera system 430, are acquired and analyzed to determine the lateral alignment between eye 470 and eyepiece lens assembly 435. These initial alignment images may be illuminated with infrared (IR) light output from illuminator 405 (or an independent illuminator associated with alignment tracking camera system 430) so as not to trigger an iris constriction response, which narrows the imaging path to retina 475. In other embodiments, conventional white light or other chromatic light is used to acquire the initial alignment images. The initial alignment image is then analyzed by controller 415 to identify any misalignment, reposition an eye location reference within dynamic fixation image 427 to encourage appropriate eye positioning relative to the selected eyebox, and then trigger acquisition of one or more subsequent eye images (e.g., retinal image burst) with image sensor 410. The subsequent images may be full color images, specific chromatic images, or even IR images as desired.

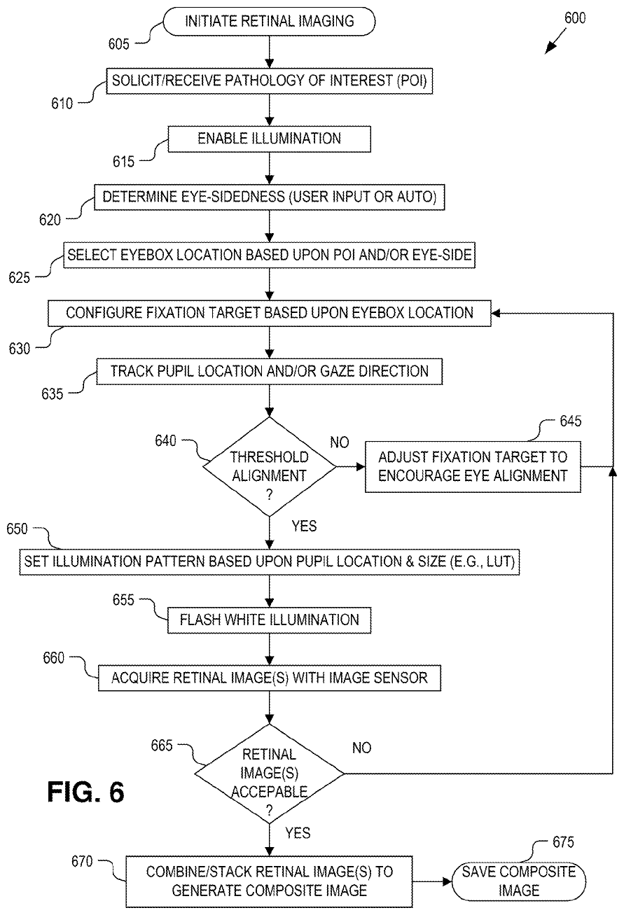

FIG. 6 is a flow chart illustrating a process 600 for capturing retinal images using dynamic eyebox locations, fixation targets, and illumination patterns based upon eye sidedness and/or pathologies of interest, in accordance with an embodiment of the disclosure. The order in which some or all of the process blocks appear in process 600 should not be deemed limiting. Rather, one of ordinary skill in the art having the benefit of the present disclosure will understand that some of the process blocks may be executed in a variety of orders not illustrated, or even in parallel.

In a process block 605, the retinal imaging process is initiated. Initiation may include the user pressing a power button on user interface 420. After powering on, controller 415 obtains an indication of the POI related to the eye being examined. This indication may be solicited via user interface 420, or otherwise input by the user/operator of retinal imaging system 400. Example POIs may include diabetic retinopathy, glaucoma, or otherwise. Determination of the particularly POI enables controller 405 to configure the eyebox location and/or illumination patterns to best inspect the portion(s) of retina 475 that is/are most pertinent to the particular ophthalmic disease selected.

In a process block 615, illumination is enabled to obtain preliminary eye images to facilitate eye tracking and/or determine eye-sidedness. In one embodiment, this initial illumination is IR illumination output from alignment tracking camera system 430 and/or IR emitters of light sources 505. The IR illumination reduces the likelihood that the light will result in a physiological response that constricts the iris prior to acquiring the primary retinal images.

In a process block 620, the eye-sidedness (i.e., right-sided eye or a left-sided eye) is determined. Eye-sidedness may be manually input via user interface 420 or automatically determined by controller 415 based upon image analysis and feature identification performed on a preliminary image of the eye. The preliminary image may be an IR retinal image acquired via image sensor 410 and/or eye images acquired by alignment tracking camera system 430.

With one or both of eye-sidedness and POI determined, the eyebox location for retinal imaging system 400 may be selected (process block 625). The determination may be based upon either one or both of these factors. The eyebox location is the location of the eyebox of the imaging system, which is a bound region in space defined relative to the eyepiece lens assembly. As illustrated in FIGS. 3A and 3B, the eyebox location for the right eye will generally be offset to the left (e.g., offset left approximately 1.5 mm) while the eyebox location for the left eye will generally be offset to the right (e.g., offset right approximately 1.5 mm). Identification of a particular POI may be used to further refine this eyebox location in addition to the eye-sided defaults. For example, an identification of diabetic retinopathy will translate the eyebox location to center the retinal image over macula 205 for the given eye-side while an identification of glaucoma will translate the eyebox location to center the retinal image over optic disc 215 for the given eye-side. Of course, other translations and eye locations may be focused upon dependent upon the POI. Additionally (or alternatively), these pathology-based offsets serve to move image artifacts away from the retinal regions of interest as determined based upon the POI.

With the eyebox location selected, the fixation location of dynamic fixation target 425 may be configured to encourage eye 470 to adjust its position and/or gaze direction accordingly (process block 630). FIGS. 7A and 7B illustrate example dynamic fixation images 705 and 710, respectively, output from display 426. The dynamic fixation images 705 and 710 both include an eyebox reference 715 and an eye location reference 720. Eyebox reference 715 is a virtual marker on display 426 that is positioned on display 426 based upon the selected eyebox location and represents the eyebox itself. Eye location reference 720 is a virtual marker on display 426 that represents the patient's pupil and its position on display 426 changes in real-time as the user attempts to align their eye with eyepiece lens 435. In other words, the position of eye location reference 720 tracks the eye location relative to eyepiece lens 435 based upon output from alignment tracking camera system 430 or image sensor 410 (process block 635). In various embodiments, alignment tracking camera system 430 may be used for gross eye alignment based upon pupil/iris tracking while image sensor 410 may be used for fine eye alignment based upon retinal tracking. Dynamic fixation images 705 and 710 may operate as a sort of game where the patient is told to concentrically align the two circular markers by moving their eye relative to eyepiece lens assembly 435. Eyebox alignment is achieved when the eye location reference 720 is moved into eyebox reference 714 (decision block 640), as illustrated in FIG. 7B. By dynamically moving the eye location reference 720, the fixation target is adjusted and the user is encouraged or coaxed into alignment (process block 645) as they attempt to concentrically align the reference markers. Of course, other alignment/fixation images may be implemented to encourage the necessary threshold alignment for obtaining a satisfactory retinal image.

As threshold alignment is achieved (decision block 640), illuminator 405 is configured by controller 415 to select the appropriate illumination pattern for retinal imaging. The illumination pattern may be selected based upon pupil location and pupil size to reduce image artifacts and optimize retinal image quality (process block 650). In one embodiment, a lookup table (LUT) may index illumination patterns to pupil position and/or pupil size. In yet other embodiments, the LUT may further index illumination patterns to POI and/or eye sidedness for further pattern refinement. For example, the illumination pattern may not only consider the current location of the eye relative to eyepiece lens assembly 435, but also the anatomical feature that is relevant to a given pathology and thus select an illumination pattern that shifts various image artifacts away from that anatomic feature in the retinal images. This may be considered a finer illumination pattern refinement in addition to the selection of the illumination pattern based upon real-time eye position tracking.

With threshold alignment achieved (decision block 640) and the appropriate illumination pattern selected (process block 650), illuminator 405 illuminates retina 475 through the pupil. This illumination may be a white light flash, though the particular wavelengths used for illumination (e.g., broadband white light, IR light, near-IR, etc.) may be tailored for a particular pathology or application. The illumination flash in process block 655 may only last for a period of time (e.g., 200 msec) that is less than or equal to the human physiological response time (e.g., pupil constriction or eye blink). While illumination is active, one or more retinal images are acquired (process block 660). In one embodiment, acquisition of a burst of retinal images (e.g., 5, 10, 20, 50, 100 images) is triggered during the illumination window and while the eye remains in the selected eyebox as determined from real-time feedback from alignment tracking camera system 430 (or image sensor 410).

The burst of retinal images may be buffered onboard a camera chip including image sensor 410 where an image signal processor (ISP) can quickly analyze the quality of the acquired retinal images. The ISP may be considered a component of controller 415 (e.g., decentralized offload compute engine) that is located close to image sensor 410 to enable highspeed image processing. If the images are occluded, obscured, or otherwise inadequate, then process 600 returns to process block 630 to repeat the relevant portions of process 600. However, if the acquired images are collectively deemed sufficient to adequately capture the region of interest relevant to the POI, then the retinal images may be combined (process block 670) to generate, output, and save a high quality composite image (process block 675). A variety of different combining techniques may be implemented such as image stacking or otherwise.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A retinal imaging system, comprising:
an eyepiece lens assembly;
an image sensor adapted to acquire a retinal image of an eye through the eyepiece lens assembly; and
a controller communicatively coupled to the image sensor, the controller including logic that when executed causes the retinal imaging system to perform operations including:
obtaining an indication of a pathology of interest (POI) related to the eye;
selecting an eyebox location for an eyebox of the retinal imaging system based at least in part on the POI, wherein the eyebox corresponds to a bound region in space defined relative to the eyepiece lens assembly; and
acquiring the retinal image of the eye when the eye is determined to be positioned within the eyebox.

2. The retinal imaging system of claim 1, wherein the controller includes further logic that when executed causes the retinal imaging system to perform additional operations including:
determining whether a sidedness of the eye is either a right-sided eye or a left-sided eye; and
selecting the eyebox location based at least in part on both the POI and the sidedness of the eye.

3. The retinal imaging system of claim 2, wherein the eyebox location is different for the right-sided eye than the left-sided eye.

4. The retinal imaging system of claim 2, wherein the sidedness of the eye is determined based at least in part upon manual user input.

5. The retinal imaging system of claim 2, wherein the sidedness of the eye is automatically determined by the retinal imaging system based at least in part upon a preliminary image of the eye.

6. The retinal imaging system of claim 2, further comprising an illuminator coupled to the controller and positioned to illuminate the eye, wherein the controller includes further logic that when executed causes the retinal imaging system to perform additional operations including:
adjusting an illumination pattern output from the illuminator based at least in part upon the POI or the sidedness of the eye.

7. The retinal imaging system of claim 6, wherein the illuminator comprises a dynamic ring illuminator that encircles an optical path extending between the eyepiece lens assembly and the image sensor to illuminate a retina of the eye through the eyepiece lens assembly.

8. The retinal imaging system of claim 1, wherein the eyebox location associated with the POI indicated as diabetic retinopathy is different than the eyebox location associated with the POI indicated as glaucoma.

9. The retinal imaging system of claim 1, further comprising a dynamic fixation target optically coupled to the eyepiece lens assembly such that the dynamic fixation target is viewable through the eyepiece lens assembly, the dynamic fixation target electrically coupled to the controller, and wherein the controller includes further logic that when executed causes the retinal imaging system to perform additional operations including:
adjusting a fixation location of the dynamic fixation target based at least in part upon the POI or the eyebox location selected for the POI.

10. The retinal imaging system of claim 9, wherein the dynamic fixation target comprises a dynamic fixation image output from a display, the dynamic fixation image comprising:
an eyebox reference rendered to a first position on the display selected based at least in part upon the POI and the eyebox location; and
an eye location reference rendered to a second position on the display based at least in part up tracking a real-time position of the eye.

11. The retinal imaging system of claim 1, further comprising an alignment tracking camera system coupled to the controller to track a real-time position of a pupil or an iris of the eye, and wherein the controller includes further logic that when executed causes the retinal imaging system to perform additional operations including:
triggering acquisition of a burst of retinal images, including the retinal image, with the image sensor when the real-time position of the pupil or the iris is determined to fall within the eyebox based upon feedback from the alignment tracking camera system.

12. A method of imaging a retina of an eye with a retinal imaging system, the method comprising:
determining whether a sidedness of the eye is either a right-sided eye or a left-sided eye;
selecting an eyebox location for an eyebox of the retinal imaging system based at least in part on the sidedness, wherein the eyebox corresponds to a bound region in space defined relative to the eyepiece lens assembly; and
acquiring the retinal image of the eye when the eye is determined to be positioned within the eyebox.

13. The method of claim 12, further comprising:
obtaining an indication of a pathology of interest (POI) related to the eye; and
selecting the eyebox location based at least in part on both the POI and the sidedness of the eye.

14. The method of claim 13, further comprising:
adjusting an illumination pattern for illuminating the eye based at least in part upon the sidedness of the eye and the POI.

15. The method of claim 14, wherein the illuminator comprises a dynamic ring illuminator that encircles an optical path extending between the eyepiece lens assembly and the image sensor to illuminate the retina of the eye through the eyepiece lens assembly.

16. The method of claim 13, further comprising:
adjusting a fixation location of a dynamic fixation image viewable through the eyepiece lens assembly based upon at least one of the POI or the eye sidedness.

17. The method of claim 16, wherein adjusting the fixation location of the dynamic fixation image comprises:
displaying an eyebox reference to a first position within the dynamic fixation image, the first position selected based upon at least one of the POI or the eye sidedness; and
displaying an eye location reference to a second position within the dynamic fixation image, the second position determined based at least in part up tracking a real-time position of the eye.

18. The method of claim 12, wherein the sidedness of the eye is determined based at least in part upon manual user input.

19. The method of claim 12, wherein the sidedness of the eye is automatically determined by the retinal imaging system based at least in part upon a preliminary image of the eye.

20. The method of claim 12, further comprising:
tracking a real-time position of a pupil or an iris of the eye with an alignment tracking camera system distinct from the image sensor; and
triggering acquisition of a burst of retinal images, including the retinal image, with the image sensor when the real-time position of the pupil or the iris is determined to fall within the eyebox based upon feedback from the alignment tracking camera system.
